(19) United States
(12) Patent Application Publication
(10) Pub. No.: US 2024/0148649 A1
(43) Pub. Date: May 9, 2024

(54) ASTAXANTHIN PICKERING EMULSION WITH COLON TARGETED DELIVERY FUNCTION, AND PREPARATION METHOD AND APPLICATION METHOD THEREOF

(71) Applicant: Fujian Agriculture and Forestry University, Fuzhou (CN)

(72) Inventors: Chunhua Wu, Fuzhou (CN); Weiquan Zhong, Fuzhou (CN); Danjie Li, Fuzhou (CN); Liang Li, Fuzhou (CN); Jing Yin, Fuzhou (CN); Xinxin Zeng, Fuzhou (CN); Chen Huang, Fuzhou (CN); Jie Pang, Fuzhou (CN)

(21) Appl. No.: 18/499,240

(22) Filed: Nov. 1, 2023

(30) Foreign Application Priority Data
Nov. 4, 2022 (CN) 202211374692.7

(51) Int. Cl. A61K 9/107; A61K 31/122; A61K 47/18; A61K 47/36; A61K 47/46

(52) U.S. Cl. CPC A61K 9/107; A61K 31/122; A61K 47/18; A61K 47/36; A61K 47/46

(57) ABSTRACT

The technical field of active substance preparations is belonged to, and provided are astaxanthin pickering emulsion with colon targeted delivery function, and a preparation method and application method thereof. The preparation method includes the following steps: S1, reparation of deacetylated-oxidized chitin nanofiber (DE-TO-ChNF)/carboxymethyl konjac glucomannan (CMKGM) nanogel; S2, preparation of astaxanthin oil phase; and S3, preparation astaxanthin pickering emulsion. Natural polysaccharide derivatives DE-TO-ChNF and CMKGM are used as raw materials, and stable crosslinking between covalent bonds and non-covalent bonds are formed between molecules in a composite system through the NHS/EDC, so as to form a stable nanogel. Then the nanogel is used as emulsifier and flaxseed oil rich in astaxanthin is used as oil phase to prepare the astaxanthin pickering emulsion by homogenization.

### BACKGROUND

Astaxanthin is a natural carotenoid, which exists in aquatic animals such as shrimp, crab and salmon. The astaxanthin has excellent antioxidant capacity and is widely used in cosmetics, health care products and pharmaceutical industries. In addition, it has been reported that the astaxanthin can prevent or treat some diseases, which include cataracts, inflammation and cardiovascular diseases. Although the astaxanthin has many functional properties, its application is greatly limited due to its easy degradation, poor water solubility and low bioavailability.

In order to improve the application of the astaxanthin, more and more attention has been paid to a packaging technology of the astaxanthin. Many delivery systems (such as emulsions, nanoparticles, and nanogels) have been applied to improve water solubility and stability of the astaxanthin, and delivery systems with colon targeted delivery characteristics can improve the bioavailability of the astaxanthin. Emulsion is a widely used transmission system at present, and pickering emulsion is a novel type of emulsion stabilized by solid nanoparticles instead of traditional emulsifier. Compared with traditional emulsion, the pickering emulsion has the advantages of low production cost, good storage stability, and unique and controllable rheological properties. In particular, pickering emulsion stabilized by natural colloidal nanoparticles such as polysaccharide and protein shows better safety and environmental protection, and has great potential for sustainable development.

deacetylated-oxidized chitin nanofiber (DE-TO-ChNF) and carboxymethyl konjac glucomannan (CMKGM) are derivatives of natural polysaccharides, and have unique characteristics. The DE-TO-ChNF has amino and carboxyl groups, and the DE-TO-ChNF is positively charged under an acidic condition and is negatively charged under an alkaline condition, and has potential of hydrogen (pH) controllability. The CMKGM is negatively charged, and has good water solubility and swelling rate. Further, the CMKGM cannot be hydrolyzed by digestive enzymes of gastrointestinal tract, but can be degraded by enzymes of colon bacteria, therefore, the CMKGM is a promising colon-targeted carrier. Both the DE-TO-ChNF and the CMKGM have great application potential in the packaging and delivery of bioactive substances.

At present, many studies have reported preparation methods of astaxanthin emulsion, but there are still some shortcomings. Chinese patent application CN201410665629.8 (with the patent publication number CN104352434A) discloses a high-stability astaxanthin ester self-microemulsion and a preparation method thereof. However, in a preparation process thereof, ethyl acetate, tween, ethanol and other reagents are used, which is not conducive to the healthy and green development of products and is difficult to be recognized by consumers. Chinese patent application CN201610375390.X (with the patent publication number CN105832569A) also has above problems, and a content of astaxanthin in astaxanthin emulsion prepared by the method thereof is lower, and a delivery efficiency is uncertain. Therefore, it is of great significance to develop healthy, green and effective astaxanthin emulsion for broadening the application of the astaxanthin.

### SUMMARY

Objectives of the disclosure are to provide astaxanthin pickering emulsion, which is healthy and green, and has colon targeted delivery function, and a preparation method and an method application thereof. According to the disclosure, the DE-TO-ChNF and the CMKGM are used as raw materials, and stable crosslinking between covalent bonds and non-covalent bonds are formed between molecules in a composite system through the NHS/EDC, so as to form a stable nanogel. Then the nanogel is used as emulsifier and flaxseed oil rich in astaxanthin is used as oil phase to prepare the astaxanthin pickering emulsion, which can greatly improve the bioavailability of astaxanthin and effectively broaden an application range of the astaxanthin in food, medicine and other fields.

In order to achieve the above objectives, the disclosure provides the following technical solutions.

An embodiment of the disclosure provides astaxanthin pickering emulsion with colon targeted delivery function, and a preparation method and application method thereof. The astaxanthin pickering emulsion with colon targeted delivery function includes the following steps:

S1, preparation of DE-TO-ChNF/CMKGM nanogel, including:

adding N-Hydroxysuccinimide (NHS) and 1-(3-Dimethylaminopropyl)-3-ethylcarbodiimide hydrochloride (EDC) into a first CMKGM solution to activate a carboxyl group and thus obtain a suspension of DE-TO-ChNF; dropping a second CMKGM solution with the same volume as that of the first CMKGM solution into the suspension of DE-TO-ChNF under magnetic stirring, to form a nanogel; performing a centrifugation process on the nanogel at a speed of 10,000 revolutions per minute (rpm) per minute (min) for 30 min, to collect DE-TO-ChNF/CMKGM nanogel; repeatedly washing the collected DE-TO-ChNF/CMKGM nanogel by using ethanol and distilled water; and freeze-drying the washed DE-TO-ChNF/CMKGM nanogel;

S2, preparation of astaxanthin oil phase, including:

weighting astaxanthin powder and dispersing the astaxanthin powder in linseed oil, and then performing heating and ultrasonic treatment on the astaxanthin powder dispersed in the linseed oil to obtain the astaxanthin oil phase with a concentration of 12 mg/mL; and

S3, preparation astaxanthin pickering emulsions, including:

dispersing the DE-TO-ChNF/CMKGM nanogel finally obtained in S1 in different water to obtain different solutions with different nanogel concentrations, and adjusting a potential of hydrogen (pH) of each of the different solutions, mixing each of the different solutions after adjusting the pH with the astaxanthin oil phase to obtain different mixtures, and homogenizing the different mixtures to thereby obtain the astaxanthin pickering emulsions.

In an embodiment, in S1, a concentration of each of the first CMKGM solution, the second CMKGM solution, and the suspension of DE-TO-ChNF is 0.25 wt %, and a pH of each of the first CMKGM solution, the second CMKGM solution, and the suspension of DE-TO-ChNF is 4.5.

In an embodiment, in S1, a molar concentration of each of the NHS and the EDC is 10 millimoles per liter (mmol/L).

In an embodiment, in S2, a temperature for the heating is 50 degrees centigrade (° C.), and a period for the heating is 2 hours (h).

In an embodiment, in S2, a period for the ultrasonic treatment is 2 min.

In an embodiment, in S3, the different nanogel concentrations are 0.5% weights by volume (w/v), 1.0% w/v, 1.5% w/v, and 2%, respectively.

In an embodiment, in S3, a volume ratio of each of the different solutions after adjusting the pH and the astaxanthin oil phase is 7:3.

In an embodiment, in S3, a temperature for the homogenizing is 10° C., a speed for the homogenizing is 12,000 rmp/min, and a period for the homogenizing is 3 min.

Compared with the related art, the disclosure at least the following advantages.
(1) The DE-TO-ChNF and the CMKGM used in the disclosure are both natural polymer products, and thus raw materials are cheap and easily available, good biocompatibility and biodegradability, environmental protection and economy.
(2) The nanogel formed by cross-linking the DE-TO-ChNF and the CMKGM in the disclosure, acting as an emulsifier, has a unique emulsifying advantage. Nanogel particles have unique rheological and interfacial properties, which can improve the stability of emulsion. Moreover, microgel nanogel particles can be well adsorbed on an oil-water interface of the emulsion. When a topological network structure inside a polymer interacts with other adsorbed particles, microgel particles will be deformed and rearranged, and an interface layer will become thicker and have mechanical elasticity due to the high combination between particles, thereby further improving the stability of the pickering emulsion.
(3) The crosslinking agent EDC/NHS used in the disclosure is nontoxic and biocompatible. The EDC/NHS can help to form amide bonds in the cross-linking process, but it does not become a part of actual cross-linking, and by-products formed in the cross-linking process can be washed with water, so there is no risk of safety and pollution.
(4) The astaxanthin pickering emulsion prepared in disclosure has a colon targeted delivery function. The CMKGM is not degraded by enzymes located in a stomach and small intestine in vivo, but can be degraded by β-mannanase located in a colon, therefore, the prepared astaxanthin pickering emulsion has a lower release amount in gastrointestinal fluid, but has a higher release amount in colon fluid containing the β-mannanase. As such, the prepared astaxanthin pickering emulsion has the specificity of colon targeted enzyme degradation, such that it can significantly improve the bioavailability of the astaxanthin and broaden the application range of astaxanthin in food and medicine fields.

### BRIEF DESCRIPTION OF DRAWINGS

FIG. 1 illustrates a flow chart of a preparation process of astaxanthin pickering emulsion of the disclosure.

FIG. 2 illustrates a cross-linking mechanism diagram of a preparation of nanogel in the disclosure.

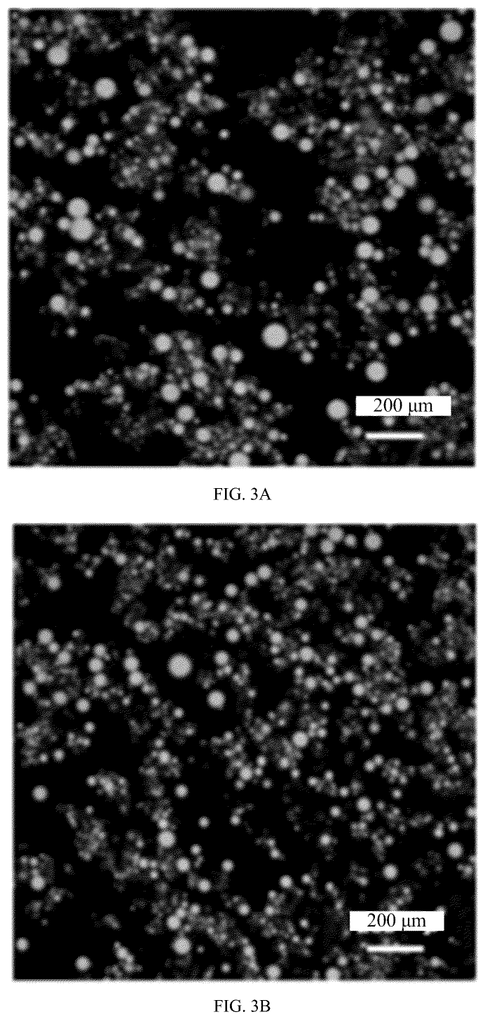

FIG. 3A-3D illustrate fluorescence micrographs of astaxanthin pickering emulsions of the disclosure.

### DETAILED DESCRIPTION OF EMBODIMENTS

Technical solutions of the disclosure will be further described hereinafter in detail with accompanying drawings and specific embodiments.

Preparation methods of required materials and solutions in the disclosure are as follows.

A preparation method of DE-TO-ChNF includes: placing 5 grams (g) of chitin powder in 250 milliliters (ml) of NaOH solution with a concentration of 33% weights by volume (w/v), adding 15 mg of sodium borohydride into the NaOH solution containing the chitin powder, and then performing magnetic stirring in a water bath at a temperature of 90 degrees centigrade (° C.) for 8 hours (h) to obtain a first solution; performing suction filtration and washing on the first solution to obtain a first filter residue, adding the first filter residue into 500 mL of sodium phosphate buffer (pH =6.8) in a flask, adding 0.08 g of 2,2,6,6-tetramethylpiperidine-1-oxygen radical (TEMPO), 6.78 g of sodium chlorite, and 0.37 g of sodium hypochlorite solution into the sodium phosphate buffer, immediately plugging the flask and performing magnetically stirring in a water bath at a temperature of 60° C. for 8 h; after reaction, performing suction filtration and washing to obtain a second filter residue, placing the second filter residue in a proper amount of deionized water to obtain a second solution, adjusting a pH of the second solution to about 3, performing a ultrasonic treatment on the second solution after adjusting the pH for 0.5 h, and freeze-dring the second solution after the ultrasonic treatment to thereby obtain the DE-TO-ChNF.

A preparation method of the CMKGM includes: dissolving 5 g of konjac powder in 50 ml of aqueous ethanol with a concentration of 85% volume in volume (v/v) to obtain a konjac solution; adding 5 g of chloroacetic acid into the konjac solution, and then performing magnetic stirring for 1.5 h with a temperature controlled at 25° C. to obtain a mixed system; heating the mixed system to a temperature of 50° C., and dropping 6 g of NaOH (dissolved in 30 ml of ethanol with a concentration of 85% (v/v)) to the mixed system for alkalization (4 h); after the reaction, performing vacuum filtration to obtain a filter residue, washing the filter residue several times by using ethanol solutions with different gradients to obtain a sample; and drying and grinding the sample at a temperature of 60° C. to thereby obtain the CMKGM.

A preparation method of simulated gastric juice includes: taking 6 ml of concentrated hydrochloric acid, adjust a volume of the concentrated hydrochloric acid to 1 liter (L) by using deionized water to obtain a solution, adjusting a pH of the solution to 1.2 by using 0.1 mol/L of HCl, and adding pepsin into the solution after adjusting the pH to make a concentration of the pepsin reach 9600 units per liter (U/L), and then filtering the solution for later use.

A preparation method of simulated small intestine fluid includes: evenly mixing 49 ml of disodium hydrogen phosphate with a concentration of 0.2 mol/L and 51 ml of sodium dihydrogen phosphate with a concentration of 0.2 mol/L to obtain a mixture; adding 800 ml of deionized water to the mixture to obtain a solution; adjusting a pH of the solution to 6.8 by using 0.1 mol/L of NaOH, and then adjusting the volume of the solution after adjusting the pH to 1 L by using deionized water; and adding trypsin to the solution after adjusting the volume to make a concentration of the trypsin reach 25,000 U/L, and then filtering the solution for later use.

A preparation of simulated colon fluid includes: evenly mixing 81 ml of disodium hydrogen phosphate with a concentration of 0.2 mol/L and 19 ml of sodium dihydrogen phosphate with a concentration of 0.2 mol/L to obtain a mixture; adding 800 ml of deionized water to the mixture to obtain a solution; adjusting a pH of the solution to 7.4 by using 0.1 mol/L of NaOH, and then adjusting the volume of the solution after adjusting the pH to 1 L by using deionized water; and adding β-mannanase to the solution after adjusting the volume to make a concentration of the β-mannanase reach 600 U/L, and then filtering the solution for later use.

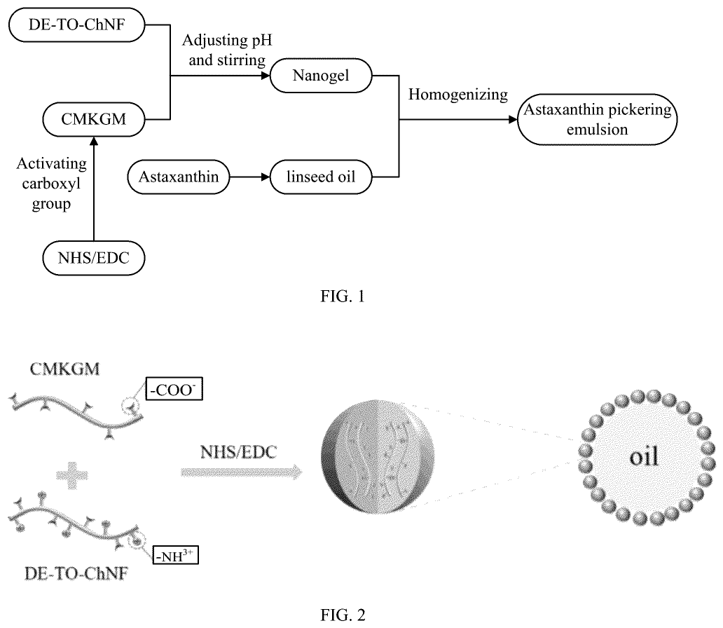

As shown in FIG. 1, a preparation method of astaxanthin pickering emulsion with colon targeted delivery function includes the following steps.

In S1, DE-TO-ChNF/CMKGM nanogel is prepared, and a process thereof includes: adding N-Hydroxysuccinimide (NHS) and 1-(3-Dimethylaminopropyl)-3-ethylcarbodiimide hydrochloride (EDC) into a first CMKGM solution to activate a carboxyl group and thus obtain a suspension of DE-TO-ChNF; dropping a second CMKGM solution with the same volume as that of the first CMKGM solution into the suspension of DE-TO-ChNF under magnetic stirring, to form a nanogel; performing a centrifugation process on the nanogel at a speed of 10,000 revolutions per minute (rpm) per minute (min) for 30 min, to collect DE-TO-ChNF/CMKGM nanogel; repeatedly washing the collected DE-TO-ChNF/CMKGM nanogel by using ethanol and distilled water; and freeze-drying the washed DE-TO-ChNF/CMKGM nanogel.

In S2, astaxanthin oil phase is prepared, and a process thereof includes: weighting astaxanthin powder and dispersing the astaxanthin powder in linseed oil, and then performing heating and ultrasonic treatment on the astaxanthin powder dispersed in the linseed oil to obtain the astaxanthin oil phase with a concentration of 12 mg/mL.

In S3, astaxanthin pickering emulsions are prepared, and a process thereof includes: dispersing the DE-TO-ChNF/CMKGM nanogel finally obtained in S1 in different water to obtain different solutions with different nanogel concentrations, and adjusting a potential of hydrogen (pH) of each of the different solutions, mixing each of the different solutions after adjusting the pH individually with the astaxanthin oil phase to obtain different mixtures, and homogenizing the different mixtures to thereby obtain the astaxanthin pickering emulsions.

In an embodiment, in S1, a concentration of each of the first CMKGM solution, the second CMKGM solution, and the suspension of DE-TO-ChNF is 0.25 wt %, and a pH of each of the first CMKGM solution, the second CMKGM solution, and the suspension of DE-TO-ChNF is 4.5.

In an embodiment, in S1, a molar concentration of each of the NHS and the EDC is 10 millimoles per liter (mmol/L).

In an embodiment, in S2, a temperature for the heating is 50 degrees centigrade (° C.), and a period for the heating is 2 hours (h).

In an embodiment, a period for the ultrasonic treatment is 2 min.

In an embodiment, in S3, the different nanogel concentrations are 0.5% weights by volume (w/v), 1.0% w/v, 1.5% w/v, and 2%, respectively.

In an embodiment, in S3, a volume ratio of each of the different solutions after adjusting the pH and the astaxanthin oil phase is 7:3.

In an embodiment, in S3, a temperature for the homogenizing is 10° C., a speed for the homogenizing is 12,000 rmp/min, and a period for the homogenizing is 3 min.

The technical solutions of the disclosure will be described clearly and completely hereinafter in combination with the embodiments in the disclosure. It is apparent that the described embodiments are only a part of embodiments of the disclosure, not the whole embodiments. Based on the descried embodiments in the disclosure, all other embodiments obtained by ordinary technicians in the field without creative labor belong to the scope of protection of the disclosure.

### Embodiment 1

### S1, Preparation of (DE-TO-ChNF/ CMKGM Nanogel

NHS and EDC are firstly added into a first CMKGM solution to activate a carboxyl group and thus obtain a suspension of DE-TO-ChNF; a second CMKGM solution with the same volume as that of the first CMKGM solution is dropped into the suspension of DE-TO-ChNF under magnetic stirring, to form a nanogel; a centrifugation process is performed on the nanogel at a speed of 10,000 revolutions per minute (rpm) per minute (min) for 30 min, to collect DE-TO-ChNF/CMKGM nanogel; the collected DE-TO-ChNF/CMKGM nanogel is repeatedly washed by using ethanol and distilled water; and the washed DE-TO-ChNF/CMKGM nanogel is freeze-dryed.

### S2, Preparation of Astaxanthin Oil Phase

Astaxanthin powder is weighted and the astaxanthin powder is dispersed in linseed oil, and then heating and ultrasonic treatment is performed on the astaxanthin powder dispersed in the linseed oil to obtain the astaxanthin oil phase with a concentration of 12 mg/mL.

### S3, Preparation of Astaxanthin Pickering Emulsion

The DE-TO-ChNF/CMKGM nanogel finally obtained in S1 is dispersed in water to obtain a solution with a nanogel concentration of 0.5%, and a pH of the solution is adjusted, the solution after adjusting the pH is mixed with the astaxanthin oil phase to obtain a mixture, and the mixture is homogenized to thereby obtain the astaxanthin pickering emulsion.

### Embodiment 2

### S1, Preparation of (DE-TO-ChNF/ CMKGM Nanogel

NHS and EDC are firstly added into a first CMKGM solution to activate a carboxyl group and thus obtain a suspension of DE-TO-ChNF; a second CMKGM solution with the same volume as that of the first CMKGM solution is dropped into the suspension of DE-TO-ChNF under magnetic stirring, to form a nanogel; a centrifugation process is performed on the nanogel at a speed of 10,000 revolutions per minute (rpm) per minute (min) for 30 min, to collect DE-TO-ChNF/CMKGM nanogel; the collected DE-TO-ChNF/CMKGM nanogel is repeatedly washed by using ethanol and distilled water; and the washed DE-TO-ChNF/CMKGM nanogel is freeze-dryed.

### S2, Preparation of Astaxanthin Oil Phase

Astaxanthin powder is weighted and the astaxanthin powder is dispersed in linseed oil, and then heating and ultrasonic treatment is performed on the astaxanthin powder dispersed in the linseed oil to obtain the astaxanthin oil phase with a concentration of 12 mg/mL.

### S3, Preparation of Astaxanthin Pickering Emulsion

The DE-TO-ChNF/CMKGM nanogel finally obtained in S1 is dispersed in water to obtain a solution with a nanogel concentration of 1%, and a pH of the solution is adjusted, the solution after adjusting the pH is mixed with the astaxanthin oil phase to obtain a mixture, and the mixture is homogenized to thereby obtain the astaxanthin pickering emulsion.

### Embodiment 3

### S1, Preparation of (DE-TO-ChNF/CMKGM Nanogel

NHS and EDC are firstly added into a first CMKGM solution to activate a carboxyl group and thus obtain a suspension of DE-TO-ChNF; a second CMKGM solution with the same volume as that of the first CMKGM solution is dropped into the suspension of DE-TO-ChNF under magnetic stirring, to form a nanogel; a centrifugation process is performed on the nanogel at a speed of 10,000 revolutions per minute (rpm) per minute (min) for 30 min, to collect DE-TO-ChNF/CMKGM nanogel; the collected DE-TO-ChNF/CMKGM nanogel is repeatedly washed by using ethanol and distilled water; and the washed DE-TO-ChNF/CMKGM nanogel is freeze-dryed.

### S2, Preparation of Astaxanthin Oil Phase

Astaxanthin powder is weighted and the astaxanthin powder is dispersed in linseed oil, and then heating and ultrasonic treatment is performed on the astaxanthin powder dispersed in the linseed oil to obtain the astaxanthin oil phase with a concentration of 12 mg/mL.

### S3, Preparation of Astaxanthin Pickering Emulsion

The DE-TO-ChNF/CMKGM nanogel finally obtained in S1 is dispersed in water to obtain a solution with a nanogel concentration of 1.5%, and a pH of the solution is adjusted, the solution after adjusting the pH is mixed with the astaxanthin oil phase to obtain a mixture, and the mixture is homogenized to thereby obtain the astaxanthin pickering emulsion.

### Embodiment 4

### S1, Preparation of (DE-TO-ChNF/CMKGM Nanogel

NHS and EDC are firstly added into a first CMKGM solution to activate a carboxyl group and thus obtain a suspension of DE-TO-ChNF; a second CMKGM solution with the same volume as that of the first CMKGM solution is dropped into the suspension of DE-TO-ChNF under magnetic stirring, to form a nanogel; a centrifugation process is performed on the nanogel at a speed of 10,000 revolutions per minute (rpm) per minute (min) for 30 min, to collect DE-TO-ChNF/CMKGM nanogel; the collected DE-TO-ChNF/CMKGM nanogel is repeatedly washed by using ethanol and distilled water; and the washed DE-TO-ChNF/CMKGM nanogel is freeze-dryed.

### S2, Preparation of Astaxanthin Oil Phase

Astaxanthin powder is weighted and the astaxanthin powder is dispersed in linseed oil, and then heating and ultrasonic treatment is performed on the astaxanthin powder dispersed in the linseed oil to obtain the astaxanthin oil phase with a concentration of 12 mg/mL.

### S3, Preparation of Astaxanthin Pickering Emulsion

The DE-TO-ChNF/CMKGM nanogel finally obtained in S1is dispersed in water to obtain a solution with a nanogel concentration of 2%, and a pH of the solution is adjusted, the solution after adjusting the pH is mixed with the astaxanthin oil phase to obtain a mixture, and the mixture is homogenized to thereby obtain the astaxanthin pickering emulsion.

The performance of the prepared astaxanthin pickering emulsions in the embodiments 1-4 are tested.

### (1) Creaming Index

A creaming index (CI) is used to quantitatively characterize the stability of emulsion. The samples are placed in a refrigerator at a temperature of 4° C., and a height of a creaming layer and a total height of the emulsion are determined after a certain period. The CI is calculated by the following formula:

$$\\ C I ( \% ) = \frac{HL}{TH} \times 100 \%$$

where HL represents the height of the creaming layer, and TH represents the total height of the emulsion.

### (2) Droplet Particle Size

A droplet particle size of each emulsion is determined by a malvin particle size analyzer at room temperature.

### (3) Release Rate In Vitro

The emulsions are placed in the simulated gastric juice, the simulated intestinal juice and the simulated colon juice at a temperature of 37° C. and shaken at a speed of 150 rpm/min for 3 hours, and then release rates of astaxanthin are detected.

### (4) Morphological Observation

A microscopic morphology of each sample is recorded using a confocal laser scanning microscope. Before preparing the emulsion, the astaxanthin oil phase and the DE-TO-ChNF/CMKGM nanogel are labeled with Nile red (0.5 mg/ml) and Nile blue (1 mg/ml) respectively. In addition, excitation wavelengths of the Nile red and the Nile blue are set to 488 nanometers (nm) and 633 nm, respectively.

Parts of experimental results are as follows in tables 1-3.

TABLE 1
Creaming indexes of emulsions prepared under different nanogel concentrations

| | | Creaming | Creaming index | Creaming index | Creaming index |
|---|---|---|---|---|---|
| | | index (%) | (%) of emulsion | (%) of emulsion | (%) of emulsion |
| | Nanogel | of fresh | after standing | after standing | after standing |
| Sample | concentration | emulsion | for one day | for one week | for one month |
| Embodiment 1 | 0.5% | 28.79 | 43.47 | 46.81 | 47.33 |
| Embodiment 2 | 1.0% | 14.29 | 30.66 | 31.61 | 31.81 |
| Embodiment 3 | 1.5% | 0 | 3.33 | 3.84 | 4.21 |
| Embodiment 4 | 2.0% | 0 | 2.36 | 2.65 | 2.71 |

TABLE 2
Droplet particle sizes of emulsions prepared
under different nanogel concentrations

| | | Droplet particle |
|---|---|---|
| Sample | Nanogel concentration | size (μm) |
| Embodiment 1 | 0.5% | 65.8 |
| Embodiment 2 | 1.0% | 47.3 |
| Embodiment 3 | 1.5% | 30.9 |
| Embodiment 4 | 2.0% | 21.2 |

TABLE 3
Release rates in vitro of emulsions prepared
under different nanogel concentrations

| | | | Release rate | |
|---|---|---|---|---|
| | | Release rate | in vitro (%) | Release rate |
| | | in vitro (%) | in the | in vitro (%) |
| | Nanogel | in the | simulated | in the |
| | concen- | simulated | intestinal | simulated |
| Sample | tration | gastric juice | juice | colon juice |
| Embodiment 1 | 0.5% | 17 | 21 | 38 |
| Embodiment 2 | 1.0% | 15 | 20 | 35 |
| Embodiment 3 | 1.5% | 12 | 16 | 32 |
| Embodiment 4 | 2.0% | 11 | 14 | 29 |

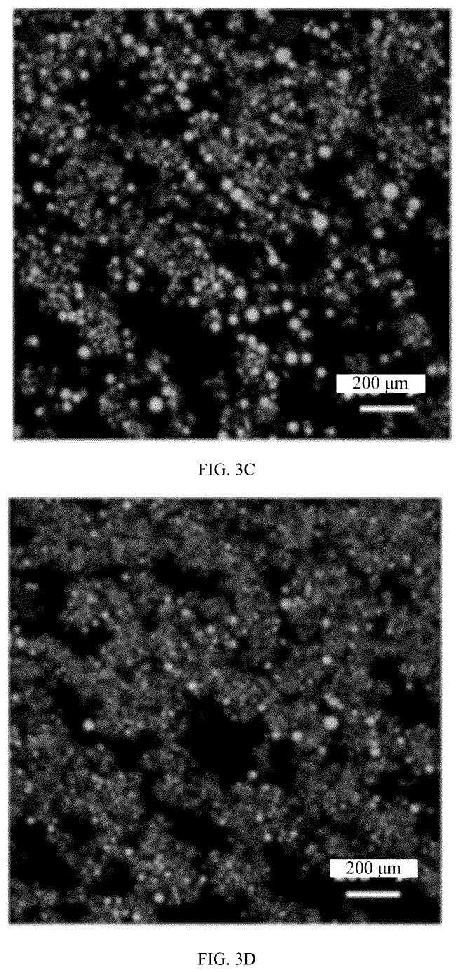

Fluorescence micrographs of astaxanthin pickering emulsions are shown in FIG. 3A-3D, which correspond to nanogel concentrations of 0.5%, 1%, 1.5%, and 2%, respectively.

The above is only the preferred embodiment of the disclosure, and it should be pointed out that a person skilled in the art can make several improvements and embellishments without departing from the principle of the disclosure, and these improvements and embellishments should also be regarded as the protection scope of the disclosure.

1. A preparation method of astaxanthin pickering emulsion with colon targeted delivery function, comprising the following steps:
(a) preparation of deacetylated-oxidized chitin nanofiber (DE-TO-ChNF)/carboxymethyl konjac glucomannan (CMKGM) nanogel, comprising:
adding N-Hydroxysuccinimide (NHS) and 1-(3-Dimethylaminopropyl)-3-ethylcarbodiimide hydrochloride (EDC) into a CMKGM solution to activate a carboxyl group and thus obtain an activated CMKGM solution; adding the activated CMKGM solution into a suspension of DE-TO-ChNF with the same volume as that of the activated CMKGM solution under magnetic stirring, to form a nanogel; performing a centrifugation process on the nanogel at a speed of 10,000 rpm/min for 30 minutes, to collect DE-TO-ChNF/CMKGM nanogel; repeatedly washing the collected DE-TO-ChNF/CMKGM nanogel by using ethanol and distilled water; and freeze-drying the washed DE-TO-ChNF/CMKGM nanogel to obtain the dryed DE-TO-ChNF/CMKGM nanogel;
(b) preparation of astaxanthin oil phase, comprising:
weighing astaxanthin powder and dispersing the astaxanthin powder in linseed oil, and then performing heating and ultrasonic treatment on the astaxanthin powder dispersed in the linseed oil to obtain the astaxanthin oil phase with a concentration of 12 mg/mL; and
(c) preparation astaxanthin pickering emulsions, comprising:
dispersing the dryed DE-TO-ChNF/CMKGM nanogel finally obtained in step (a) in water to obtain different solutions with different concentrations, and adjusting a pH of each of the different solutions, mixing each of the different solutions after adjusting the pH with the astaxanthin oil phase to obtain different mixtures, and homogenizing the different mixtures to thereby obtain the astaxanthin pickering emulsions;
wherein the DE-TO-ChNF of the suspension of DE-TO-ChNF is prepared by raw materials comprising: chitin powder, NaOH solution, sodium borohydride, sodium phosphate buffer, 2,2,6,6-tetramethylpiperidine-1 -oxygen radical (TEMPO), sodium chlorite, sodium hypochlorite solution, and deionized water.

2. The preparation method of astaxanthin pickering emulsion with colon targeted delivery function according to claim 1, wherein in step (a), a concentration of each of the CMKGM solution and the suspension of DE-TO-ChNF is 0.25 wt %, and a pH of each of the CMKGM solution and the suspension of DE-TO-ChNF is 4.5.

3. The preparation method of astaxanthin pickering emulsion with colon targeted delivery function according to claim 1, wherein in step (a), a molar concentration of each of the NHS and the EDC is 10 mmol/L.

4. The preparation method of astaxanthin pickering emulsion with colon targeted delivery function according to claim 1, wherein in step (b), a temperature for the heating is 50° C., and a period for the heating is 2 hours.

5. The preparation method of astaxanthin pickering emulsion with colon targeted delivery function according to claim 1, wherein in step (b), a period for the ultrasonic treatment is 2 minutes.

6. The preparation method of astaxanthin pickering emulsion with colon targeted delivery function according to claim 1, wherein in step (c), the different concentrations are 0.5% w/v, 1.0% w/v, 1.5% w/v, and 2% w/v, respectively.

7. The preparation method of astaxanthin pickering emulsion with colon targeted delivery function according to claim 1, wherein in step (c), a ratio of each of the different solutions after adjusting the pH and the astaxanthin oil phase is 7:3.

8. The preparation method of astaxanthin pickering emulsion with colon targeted delivery function according to claim 1, wherein in step (c), a temperature for the homogenizing is 10° C., a speed for the homogenizing is 12,000 rpm/min, and a period for the homogenizing is 3 minutes.

9. A preparation method of astaxanthin pickering emulsion with colon targeted delivery function, comprising the following steps:
adding N-Hydroxysuccinimide (NHS) and 1-(3-Dimethylaminopropyl)-3-ethylcarbodiimide hydrochloride (EDC) into a carboxymethyl konjac glucomannan (CMKGM) solution to activate a carboxyl group of the CMKGM solution and thus obtain activated CMKGM solution; adding the activated CMKGM solution into a suspension of deacetylated-oxidized chitin nanofiber (DE-TO-ChNF) with the same volume as that of the activated CMKGM solution under magnetic stirring, to form a nanogel; performing a centrifugation process on the nanogel at a speed of 10,000 rpm/min for 30 minutes, to obtain a DE-TO-ChNF/CMKGM nanogel; repeatedly washing the obtained DE-TO-ChNF/CMKGM nanogel by using ethanol and distilled water; and freeze-drying the washed DE-TO-ChNF/CMKGM nanogel to obtain the dryed DE-TO-ChNF/CMKGM nanogel;
weighing astaxanthin powder and dispersing the astaxanthin powder in linseed oil, and then performing heating and ultrasonic treatment on the astaxanthin powder dispersed in the linseed oil to obtain astaxanthin oil phase with a concentration of 12 mg/mL; and
dispersing the dryed DE-TO-ChNF/CMKGM nanogel in water to obtain different solutions with different concentrations, and adjusting a pH of each of the different solutions, mixing each of the different solutions after adjusting the pH with the astaxanthin oil phase to obtain different mixtures, and homogenizing the different mixtures to thereby obtain astaxanthin pickering emulsions;
wherein the DE-TO-ChNF of the suspension of DE-TO-ChNF is prepared by raw materials comprising: chitin powder, NaOH solution, sodium borohydride, sodium phosphate buffer, 2,2,6,6-tetramethylpiperidine-1 -oxygen radical (TEMPO), sodium chlorite, sodium hypochlorite solution, and deionized water.

10. A preparation method of astaxanthin pickering emulsion with colon targeted delivery function, comprising the following steps:
adding N-Hydroxysuccinimide (NHS) and 1-(3-Dimethylaminopropyl)-3-ethylcarbodiimide hydrochloride (EDC) into a carboxymethyl konjac glucomannan (CMKGM) solution to activate a carboxyl group of the CMKGM solution and thus obtain activated CMKGM solution;
preparing deacetylated-oxidized chitin nanofiber (DE-TO-ChNF) by raw materials comprising:
chitin powder, NaOH solution, sodium borohydride, sodium phosphate buffer, 2,2,6,6-tetramethylpiperidine-1 -oxygen radical (TEMPO), sodium chlorite, sodium hypochlorite solution, and deionized water, and dispersing the prepared DE-TO-ChNF in water to obtain a suspension of DE-TO-ChNF;
adding the activated CMKGM solution into the suspension of DE-TO-ChNF with the same volume as that of the activated CMKGM solution under magnetic stirring, to form a nanogel;
performing a centrifugation process on the nanogel at a speed of 10,000 rpm/min for 30 minutes, to obtain a DE-TO-ChNF/CMKGM nanogel; repeatedly washing the obtained DE-TO-ChNF/CMKGM nanogel by using ethanol and distilled water; and freeze-drying the washed DE-TO-ChNF/CMKGM nanogel to obtain the dryed DE-TO-ChNF/CMKGM nanogel;
weighing astaxanthin powder and dispersing the astaxanthin powder in linseed oil, and then performing heating and ultrasonic treatment on the astaxanthin powder dispersed in the linseed oil to obtain astaxanthin oil phase with a concentration of 12 mg/mL; and
dispersing the dryed DE-TO-ChNF/CMKGM nanogel in water to obtain different solutions with different concentrations, and adjusting a pH of each of the different solutions, mixing each of the different solutions after adjusting the pH with the astaxanthin oil phase to obtain different mixtures, and homogenizing the different mixtures to thereby obtain astaxanthin pickering emulsions.

11. The preparation method of astaxanthin pickering emulsion with colon targeted delivery function according to claim 10, wherein the preparing the DE-TO-ChNF comprises:
placing the chitin powder in the NaOH solution, adding the sodium borohydride into the NaOH solution containing the chitin powder, and then performing magnetic stirring in a water bath to obtain a first solution; performing suction filtration and washing on the first solution to obtain a first filter residue, adding the first filter residue into the sodium phosphate buffer in a flask, adding the TEMPO, the sodium chlorite, and the sodium hypochlorite solution into the sodium phosphate buffer, plugging the flask and performing magnetically stirring in a water bath for reacting; after reaction, performing suction filtration and washing to obtain a second filter residue, placing the second filter residue in the deionized water to obtain a second solution, adjusting a pH of the second solution, performing a ultrasonic treatment on the second solution after adjusting the pH, and freeze-dring the second solution after the ultrasonic treatment to thereby obtain the DE-TO-ChNF.
